(19) United States
(12) Patent Application Publication
(10) Pub. No.: US 2024/0101499 A1
(43) Pub. Date: Mar. 28, 2024

(54) ZEOLITE CATALYST, PROCESS FOR PREPARATION AND APPLICATION THEREOF

(71) Applicant: Council of Scientific & Industrial Research, New Dlhi (IN)

(72) Inventors: Vijay Vasant BOKADE, Maharashtra (IN); Sachin Uttamrao NANDANWAR, Maharashtra (IN); Prashant Suresh NIPHADKAR, Maharashtra (IN)

(21) Appl. No.: 18/272,507

(22) PCT Filed: Jan. 14, 2022
(86) PCT No.: PCT/IN2022/050031

§ 371 Date: Jul. 14, 2023

(30) Foreign Application Priority Data
Jan. 15, 2021 (IN) 202111002070

(51) Int. Cl. C07C 41/09; B01J 35/02; B01J 35/10; B01J 37/00; B01J 37/03; B01J 37/08; C01B 39/48

(52) U.S. Cl. CPC C07C 41/09; B01J 35/026; B01J 35/1019; B01J 35/1023; B01J 35/1038; B01J 35/1071; B01J 37/009; B01J 37/033; B01J 37/08; C01B 39/48; C01P 2002/72; C01P 2004/03; C01P 2004/38; C01P 2006/12; C01P 2006/14; C01P 2006/16

(57) ABSTRACT

The present invention relates to a Si/Al zeolite catalyst with cubical morphology, having pore diameter in the range of 0.5 to 0.6 μm, pore volume in the range of 0.2 to 0.3 cc/g, surface area in the range of 500 to 700 m²/g, and SiO2/Al2O3 ratio in the range of 30 to 200. The present invention also relates to a process for its preparation and its application in one step, one pot synthesis of ether.

### FIELD OF THE INVENTION

The present invention relates to a zeolite catalyst, a process for preparation and application thereof. Particularly, the present invention relates to a Si/Al zeolite catalyst with cubical morphology for one pot synthesis of ethers as a catalyst.

### BACKGROUND AND PRIOR ART OF THE INVENTION

Ethers, such as dimethyl ether and methyl tert-butyl ether, are known as attractive candidates for fuel additives because of their ability to reduce soot formation during the combustion process. Dimethoxy ethane, known as ethylene glycol dimethyl ether, attracts increasing interest in recent years because of its advantageous properties (high energy density and cetane number). It also shows excellent solubility, widely used as green solvent and good etherification agent in cosmetics, perfumes, pharmaceuticals and especially applied in batteries and electrolyte.

Glycol ethers, which are also commonly known as glymes, are used as aprotic solvents in a variety of applications. Glymes can be produced by a variety of methods, but are conventionally produced in commercial quantities via the Williamson synthesis or via a reaction that involves the cleavage of epoxides.

In the Williamson synthesis, a monoalkyl polyalkylene glycol is treated with a base or an alkali metal, typically molten Sodium, to form an alkoxide ion, which is then reacted with an alkyl halide such as methyl chloride to form the glyme. The by-products from the Williamson synthesis are hydrogen gas and a salt.

US2004044253A1 discloses a method of producing glycol ethers which are also commonly known as glymes. The method includes contacting a glycol with a monohydric alcohol in the presence of a polyperfluoro sulfonic acid resin catalyst under conditions effective to produce the glyme. The method can be used to produce, for example, monoglyme, ethyl glyme, diglyme, ethyl diglyme, triglyme, butyl diglyme, tetraglyme, and their respective corresponding monoalkyl ethers. The document also provides a method of producing 1,4-dioxane from mono- or diethylene glycol and tetrahydrofuran from 1,4-butanediol.

EP0186815A1 discloses process for the preparation of glycol alkyl ethers. Glycols are reacted with alkanols and/or dialkyl ethers as etherifying agents in the presence of Lewis acids as catalysts, and the glycol monoalkyl ether, glycol dialkyl ether or a mixture of the two glycol ethers are recovered from the reaction product which mainly comprises glycol monoalkyl ether and glycol dialkyl ether, unconverted glycol and unreacted etherifying agent. In this process, relatively few unusable by-products such as dioxane are formed.

All above-mentioned prior arts disclose homogeneous acid catalysts and medium or large pore zeolite, which operates either in batch or in continuous mode but not in both. These catalysts give maximum 1,2 dimethoxy ethane/glyme of 95% (polyperfluoro sulfonic acid resin) in batch process and 74% (medium and large pore zeolite) in continuous process with ethylene glycol conversion level of 90 to 96%.

In the present invention, small pore zeolite of 0.5 to 0.6 μm pore diameter having cubical morphology can use in batch as well as in continuous mode and give up to 100% selective formation of 1,2 dimethoxy ethane/glyme. The present catalyst can be used for different substrates such as ethylene glycol, 2-methoxy ethanol, propylene glycol and methanol, ethanol, propanol, octanol etc at conversion level up to 100%.

### OBJECTIVES OF THE INVENTION

The main objective of the present invention is to provide a zeolite catalyst, H-SSZ-13.

One more objective of the present invention is to provide a process for preparation of the zeolite catalyst.

Another objective of the present invention is to provide a process for etherification by using the zeolite catalyst.

### SUMMARY OF THE INVENTION

Accordingly, the present invention provides a zeolite catalyst H-SSZ-13, wherein said catalyst is characterized by a cubical morphology, pore diameter of the catalyst is in the range of 0.5 to 0.6 μm, pore volume of the catalyst is in the range of 0.2 to 0.3 cc/g, surface area of the catalyst is in the range of 500 to 700 m²/g, and SiO₂/Al₂O₃ ratio in the catalyst is in the range of 30 to 200.

In an embodiment of the present invention, said catalyst is prepared by a process comprising the steps of:
i. hydrothermally crystallizing a gel formed by fumed silica, aluminium hydroxide, sodium hydroxide, N, N, N-Trimethyladamantan-1-aminium hydroxide and water by heating at temperature in the range of 100 to 200° C. at pressure in the range of 70-120 psig for a period in the range of 4 to 9 days to obtain a slurry;
ii. filtering the slurry as obtained in step (i) followed by drying at temperature in the range of 100 to 120° C. for period in the range of 4 to 5 h to obtain a dried slurry; and
iii. calcining the dried slurry as obtained in step (ii) at temperature in the range of 500 to 600° C. for a period in the range of 10 to 14 h to afford the zeolite catalyst.

In another embodiment, present invention provides a one pot process for the synthesis of an ether comprising the step of:
reacting a first substrate with a second substrate in a molar ratio ranging between 1:1 to 1:10 in the presence of a zeolite catalyst, at a temperature in the range of 200° C. to 250° C. for a time period in the range of 2 to 7 hours to afford the ether;
wherein said process is carried out in a batch or a fixed bed continuous operation or in a continuous stirred tank reactor (CSTR).

In yet another embodiment of the present invention, there is provided a one pot process for the synthesis of an ether, wherein said first substrate is an alcohol selected from the group consisting of ethylene glycol (EG), propylene glycol, 2-methoxyethanol (MME) and 2-ethoxyethanol and the second substrate is an alcohol selected from the group consisting of methanol, ethanol, propanol and octanol.

In yet another embodiment of the present invention, said ether is selected from 1,2-dimethoxyethane (DME) or diethoxy ethane (DEE).

In yet another embodiment of the present invention, selectivity of the said ether is in the range of 30-100% and conversion of said substrate is in the range of 20-90%.

In yet another embodiment of the present invention, a binder is used in the fixed bed continuous operation, wherein content of the binder with respect to the catalyst is in the range of 0-50% and wherein said binder is selected from alumina, or silica or mixture thereof.

In yet another embodiment of the present invention, shape of said catalyst is extrudates, pellets or tablets and wherein size of the catalyst in a continuous operation is 1 mm×1 mm to 5 mm×5 mm and said catalyst is recyclable.

In yet another embodiment of the present invention, for said fixed bed continuous operation, the weight hourly space velocity (WHSV) with respect to first substrate is in the range of 0.1 to 3 hours⁻¹ and nitrogen pressure is in the range of 1 to 10 bar.

In yet another embodiment of the present invention, for batch process, loading of said catalyst is in the range of 2-10%.

### ABBREVIATION

MME: 2-methoxyethanol
EG: ethylene glycol
DME: 1,2-dimethoxyethane
DEE: diethoxy ethane
WHSV: weight hourly space velocity
CSTR: continuous stirred-tank reactor

### BRIEF DESCRIPTION OF THE DRAWINGS

FIG. 1 describes the powder XRD pattern of H-SSZ-13 catalyst.

FIG. 2 describes FESEM of H-SSZ-13 catalyst.

### DETAILED DESCRIPTION OF THE INVENTION

The present invention provides a zeolite catalyst characterized in that the catalyst possesses cubical morphology, the pore diameter is in the range of 0.5 to 0.6 μm, the pore volume is in the range of 0.2 to 0.3 cc/g, the surface area is in the range of 500 to 700 m²/g, the SiO₂/Al₂O₃ ratio is in the range of 30 to 200, wherein the zeolite catalyst is H-SSZ-13.

The present invention also provides a process for preparation of the zeolite catalyst comprising:
i. hydrothermally crystallizing a gel formed by fumed silica, aluminium hydroxide, sodium hydroxide, N, N, N-Trimethyladamantan-1-aminium hydroxide and water by heating at temperature in the range of 100 to 200° C. at pressure in the range of 70-120 psig for a period in the range of 4 to 9 days to obtain a slurry;
ii. filtering the slurry as obtained in step (i) followed by drying at temperature in the range of 100 to 120° C. for period in the range of 4 to 5 h to obtain a dried slurry; and
iii. calcining the dried slurry as obtained in step (ii) at temperature in the range of 500 to 600° C. for a period in the range of 10 to 14 h to afford the zeolite catalyst.

The zeolite catalyst of the present invention is used in the preparation of ether from alcohol.

The present invention further provides a one step, one pot process for synthesis of ether comprising:
reacting a first substrate with a second substrate in presence of the catalyst of the present invention at a temperature in the range of 200° C. to 250° C. for a time period in the range of 2 to 7 hours to afford the ether.

The first substrate is an alcohol selected from the group consisting of ethylene glycol (EG), propylene glycol, 2-methoxyethanol (MME) and 2-ethoxyethanol.

The second substrate is an alcohol selected from the group consisting of methanol, ethanol, propanol and octanol.

The ether is selected from the group consisting of 1,2-dimethoxyethane (DME) and diethoxy ethane (DEE).

The selectivity of the desired ether is in the range of 30-100%.

The conversion of the substrate is in the range of 20-90%.

The reaction can be carried out in a batch or a continuous operation in a CSTR.

The reaction can be carried out in a fixed bed continuous operation.

The molar ratio of the first substrate to the second substrate is in the range of 1:1 to 1:10, preferably 1:3 to 1:10.

A binder may be used in the continuous mode of operation to bind the catalyst powder.

The content of the binder with respect to the catalyst for continuous operation is in the range of 0.1-50%.

The binder can be alumina, or silica or a mixture thereof.

The shape of catalyst for continuous mode can be extrudates, pellets or tablets.

The catalyst size with the binder used in the continuous mode is 1 mm×1 mm to 5 mm×5 mm.

The catalyst used in the reaction for preparation of ether is a zeolite catalyst characterized in that the catalyst possesses cubical morphology, the pore diameter is in the range of 0.5 to 0.6 μm, the pore volume is in the range of 0.2 to 0.3 cc/g, the surface area is in the range of 500 to 700 m²/g, the SiO₂/Al₂O₃ ratio is in the range of 30 to 200.

The required catalyst loading in the batch process is in the range of 2 to 10%.

The catalyst used in the reaction for preparation of ether is H-SSZ-13 (SiO₂/Al₂O₃-96).

In a continuous process, weight hourly space velocity (WHSV) with respect to the first substrate is in the range of 0.1 to 3 hours⁻¹, preferably in the range of 0.7-2.5 hours⁻¹.

In a continuous process, the nitrogen pressure is required in the range of 1 to 10 bar, preferably 5 bar.

Primary Reaction Etherification to form product Dimethoxyethane

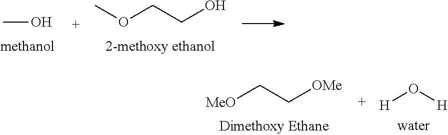

Secondary reaction: Self Etherification of 2-methoxy ethanol to form byproduct 1,4 Dioxane

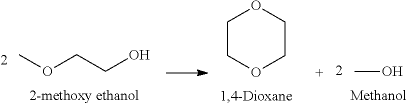

The catalyst used in the one step, one pot process for the synthesis of ether is recyclable.

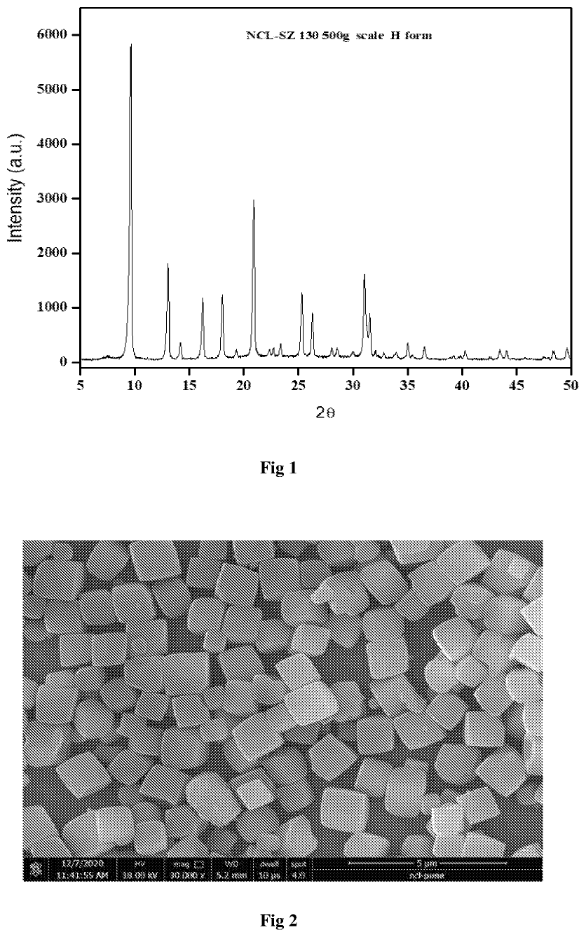

FIG. 1 describes the XRD pattern of H-SSZ-13 catalyst. In XRD, the first peak (100 plane) is more intense than the normal H-SSZ-13.

FIG. 2 describes FESEM of H-SSZ-13 catalyst. FESEM observed cubical uniform morphology in the range of 2-2.5-micron size.

Several experiments were conducted in Batch as well as in a continuous operation mode by using H-SSZ-13 catalyst for etherification. Results of the experiments are summarized in Table-1 below:

TABLE 1

| | | | | | | % | % | | |
|---|---|---|---|---|---|---|---|---|---|
| | | | | SiO₂/ | | MME/ | DME/ | % | % 1,4 |
| | | | Reaction | Al₂O₃ | Operating | EG | DEE | MME | Dioxane |
| No. | Substrate 1 | Substrate 2 | Type | ratio | parameters | Conv. | Sel. | Sel. | Sel. |
| 1 | MME | Methanol | Batch | 96 | 210° C., | 67 | 97 | — | 3 |
| | | (MeOH) | | | (MME:MeOH) | | | | |
| | | | | | molar ratio: 1:3.5, | | | | |
| | | | | | Catalyst loading: | | | | |
| | | | | | 7% w.r.t MME, | | | | |
| | | | | | Reaction time: 5 h | | | | |
| 2 | MME | MeOH | Batch | 96 | 210° C., | 66 | 95 | — | 5 |
| | | | | 1^{st} | (MME:MeOH) | | | | |
| | | | | recycle | molar ratio: 1:3.5, | | | | |
| | | | | | Catalyst loading: | | | | |
| | | | | | 7% w.r.t MME, | | | | |
| | | | | | Reaction time: 5 h | | | | |
| 3 | MME | MeOH | Batch | 96 | 210° C., | 66 | 95 | — | 5 |
| | | | | 2^{nd} | (MME:MeOH) | | | | |
| | | | | recycle | molar ratio: 1:3.5, | | | | |
| | | | | | Catalyst loading: | | | | |
| | | | | | 7% w.r.t MME, | | | | |
| | | | | | Reaction time: 5 h | | | | |
| 4 | MME | MeOH | Batch | 96 | 210° C., | 45 | 94 | — | 6 |
| | | | | | (MME:MeOH) | | | | |
| | | | | | molar ratio: 1:3.5, | | | | |
| | | | | | Catalyst loading: | | | | |
| | | | | | 7% w.r.t MME, | | | | |
| | | | | | Reaction time: 2 h | | | | |
| 5 | MME | MeOH | Batch | 96 | 210° C., | 70 | 97 | — | 3 |
| | | | | | (MME:MeOH) | | | | |
| | | | | | molar ratio: 1:3.5, | | | | |
| | | | | | Catalyst loading: | | | | |
| | | | | | 7% w.r.t MME, | | | | |
| | | | | | Reaction time: 3 h | | | | |
| 6 | MME | MeOH | Batch | 96 | 210° C., | 65 | 97 | — | 3 |
| | | | | | (MME:MeOH) | | | | |
| | | | | | molar ratio: 1:3.5, | | | | |
| | | | | | Catalyst loading: | | | | |
| | | | | | 7% w.r.t MME, | | | | |
| | | | | | Reaction time: 4 h | | | | |
| 7 | MME | MeOH | Batch | 96 | 210° C., | 67 | 97 | — | 3 |
| | | | | | (MME:MeOH) | | | | |
| | | | | | molar ratio: 1:3.5, | | | | |
| | | | | | Catalyst loading: | | | | |
| | | | | | 7% w.r.t MME, | | | | |
| | | | | | Reaction time: 6 h | | | | |
| 8 | MME | MeOH | Batch | 96 | 210° C., | 40 | 87 | — | 13 |
| | | | | | (MME:MeOH) | | | | |
| | | | | | molar ratio: 1:1, | | | | |
| | | | | | Catalyst loading: | | | | |
| | | | | | 7% w.r.t MME, | | | | |
| | | | | | Reaction time: 3 h | | | | |
| 9 | MME | MeOH | Batch | 96 | 210° C., | 52 | 90 | — | 10 |
| | | | | | (MME:MeOH) | | | | |
| | | | | | molar ratio: 1:2, | | | | |
| | | | | | Catalyst loading: | | | | |
| | | | | | 7% w.r.t MME, | | | | |
| | | | | | Reaction time: 3 h | | | | |
| 10 | MME | MeOH | Batch | 96 | 210° C., | 67 | 97 | — | 3 |
| | | | | | (MME:MeOH) | | | | |
| | | | | | molar ratio: 1:3, | | | | |
| | | | | | Catalyst loading: | | | | |
| | | | | | 7% w.r.t MME, | | | | |
| | | | | | Reaction time: 3 h | | | | |
| 11 | MME | MeOH | Batch | 96 | 210° C., | 37 | 76 | — | 24 |
| | | | | | (MME:MeOH) | | | | |
| | | | | | molar ratio: 1:3, | | | | |
| | | | | | Catalyst loading: | | | | |
| | | | | | 2% w.r.t MME, | | | | |
| | | | | | Reaction time: 3 h | | | | |
| 12 | MME | MeOH | Batch | 96 | 210° C., | 74 | 85 | — | 15 |
| | | | | | (MME:MeOH) | | | | |
| | | | | | molar ratio: 1:3, | | | | |
| | | | | | Catalyst loading: | | | | |
| | | | | | 5% w.r.t MME, | | | | |
| | | | | | Reaction time: 3 h | | | | |
| 13 | MME | MeOH | Batch | 96 | 210° C., | 68 | 97 | — | 3 |
| | | | | | (MME:MeOH) | | | | |
| | | | | | molar ratio: 1:3, | | | | |
| | | | | | Catalyst loading: | | | | |
| | | | | | 10% w.r.t MME, | | | | |
| | | | | | Reaction time: 3 h | | | | |
| 14 | EG | MeOH | Batch | 96 | 210° C., | 90 | 40 | 35 | 25 |
| | | | | | (MME:MeOH) | | | | |
| | | | | | molar ratio: 1:3, | | | | |
| | | | | | Catalyst loading: | | | | |
| | | | | | 7% w.r.t MME, | | | | |
| | | | | | Reaction time: 3 h | | | | |
| 15 | MME | MeOH | Batch | 30 | 210° C., | 50 | 87 | — | 13 |
| | | | | | (MME:MeOH) | | | | |
| | | | | | molar ratio: 1:3, | | | | |
| | | | | | Catalyst loading: | | | | |
| | | | | | 7% w.r.t MME, | | | | |
| | | | | | Reaction time: 3 h | | | | |
| 16 | MME | MeOH | Batch | 180 | 210° C., | 70 | 92 | — | 8 |
| | | | | | (MME:MeOH) | | | | |
| | | | | | molar ratio: 1:3, | | | | |
| | | | | | Catalyst loading: | | | | |
| | | | | | 7% w.r.t MME, | | | | |
| | | | | | Reaction time: 3 h | | | | |
| 17 | MME | MeOH | Batch | 200 | 210° C., | 70 | 92 | — | 8 |
| | | | | | (MME:MeOH) | | | | |
| | | | | | molar ratio: 1:3, | | | | |
| | | | | | Catalyst loading: | | | | |
| | | | | | 7% w.r.t MME, | | | | |
| | | | | | Reaction time: 3 h | | | | |
| 18 | EG | MeOH | Batch | 30 | 210° C., | 70 | 30 | 50 | 20 |
| | | | | | (EG:MeOH) | | | | |
| | | | | | molar ratio: 1:3, | | | | |
| | | | | | Catalyst loading: | | | | |
| | | | | | 7% w.r.t MME, | | | | |
| | | | | | Reaction time: 3 h | | | | |
| 19 | EG | MeOH | Batch | 180 | 210° C., | 85 | 38 | 38 | 24 |
| | | | | | (EG:MeOH) | | | | |
| | | | | | molar ratio: 1:3, | | | | |
| | | | | | Catalyst loading: | | | | |
| | | | | | 7% w.r.t MME, | | | | |
| | | | | | Reaction time: 3 h | | | | |
| 20 | EG | MeOH | Batch | 200 | 210° C., | 83 | 35 | 40 | 25 |
| | | | | | (EG:MeOH) | | | | |
| | | | | | molar ratio: 1:3, | | | | |
| | | | | | Catalyst loading: | | | | |
| | | | | | 7% w.r.t MME, | | | | |
| | | | | | Reaction time: 3 h | | | | |
| 21 | EG | Ethanol | Batch | 96 | 210° C., | 72 | 35 | 46 | 19 |
| | | | | | (EG:EtOH) molar | | | | |
| | | | | | ratio: 1:3, Catalyst | | | | |
| | | | | | loading: 7% w.r.t | | | | |
| | | | | | MME, Reaction | | | | |
| | | | | | time: 3 h | | | | |
| 22 | MME | Ethanol | Batch | 96 | 210° C., | 60 | 80 | — | 20 |
| | | | | | (MME:EtOH) | | | | |
| | | | | | molar ratio: 1:3, | | | | |
| | | | | | Catalyst loading: | | | | |
| | | | | | 7% w.r.t MME, | | | | |
| | | | | | Reaction time: 3 h | | | | |
| 23 | MME | MeOH | Continuous | 96 | 215° C., | 30 | 100 | — | — |
| | | | | | (MME:MeOH) | | | | |
| | | | | | molar ratio: 1:3, | | | | |
| | | | | | WHSV w.r.t | | | | |
| | | | | | MME 0.7 h-1, | | | | |
| | | | | | Reaction time: 5 h, | | | | |
| | | | | | Nitrogen pressure: | | | | |
| | | | | | 5 bar | | | | |
| 24 | EG | MeOH | Continuous | 96 | 215° C., | 23 | 100 | — | — |
| | | | | | (EG:MeOH) | | | | |
| | | | | | molar ratio: 1:3, | | | | |
| | | | | | WHSV w.r.t | | | | |
| | | | | | MME 0.7 h-1, | | | | |
| | | | | | Reaction time: 5 h, | | | | |
| | | | | | Nitrogen pressure: | | | | |
| | | | | | 5 bar | | | | |
| 25 | EG | MeOH | Continuous | 30 | 215° C., | 20 | 100 | — | — |
| | | | | | (EG:MeOH) | | | | |
| | | | | | molar ratio: 1:3, | | | | |
| | | | | | WHSV w.r.t | | | | |
| | | | | | MME 0.7 h-1, | | | | |
| | | | | | Reaction time: 5 h, | | | | |
| | | | | | Nitrogen pressure: | | | | |
| | | | | | 5 bar | | | | |
| 26 | MME | MeOH | Continuous | 30 | 215° C., | 27 | 100 | — | — |
| | | | | | (MME:MeOH) | | | | |
| | | | | | molar ratio: 1:3, | | | | |
| | | | | | WHSV w.r.t | | | | |
| | | | | | MME 0.7 h-1, | | | | |
| | | | | | Reaction time: 5 h, | | | | |
| | | | | | Nitrogen pressure: | | | | |
| | | | | | 5 bar | | | | |

### EXAMPLES

Following examples are given by way of illustration and therefore should not be construed to limit the scope of the invention.

### Example 1: Preparation of NCL H-SSZ-13 Catalyst (SiO₂/Al₂O₃-96)

A mixture of fumed silica (99%, 577.2 g), aluminium hydroxide (51.45% Al₂O₃, 14.62 g), sodium hydroxide (99%, 76.96 g), N,N,N-Trimethyladamantan-1-ammonium hydroxide (25% aqueous solution, 1626 g) and water (5704.66 g) was heated at a temperature 160° C. for 4 days.

### Example 2: Preparation of Initial Gel

Equipment for Gel Preparation

TABLE 2

| | |
|---|---|
| Mixing Vessel: 20 Liter bucket | Stirring: overhead |
| Beakers: 5 lit and 500 ml plastic | Type of stirrer: axial |
| beakers | radical turbine |
| Weighing Balance: Analytical | Stirrer Blade Size: 4 blades, 5 cm |
| balance & 30 kg sansui | |
| pH meter: Digital | |

### a) Preparation of Solution a

### i) Preparation of NaOH Solution

In a plastic beaker, NaOH (77.0096 g) was added into water (300 g) and stirred for 10 minutes to obtain a NaOH solution.

TABLE 3

| | | |
|---|---|---|
| NaOH = | 77.0096 g | Mixing vessel: 500 ml Plastic beaker |
| Water = | 300 g | Time: 10 minutes |
| | | RPM: 100 |

### ii) Addition of N,N,N-Trimethyladamantan-1-Aminium Hydroxide into NaOH Solution (i)

N,N,N-Trimethyladamantan-1-aminium hydroxide (1626 g) was added into the NaOH solution (i) and stirred for 5 minutes. A clear solution was obtained.

TABLE 4

| | | |
|---|---|---|
| N,N,N- = | 1626 g | Stirring Time: 5 min |
| Trimethyladamantan- | | RPM: 160 |
| 1-aminium hydroxide | | |

### iii) Preparation of Aluminium Hydroxide Solution

In a plastic beaker, Aluminium Hydroxide (14.6289 g) was added into water (300 g) and stirred for 5 minutes to obtain an aluminium hydroxide solution.

TABLE 5

| | | |
|---|---|---|
| Aluminium = | 14.6289 g | Mixing vessel: 500 mL plastic beaker |
| Hydroxide | | Stirring Time: 5 min |
| Water = | 300 g | RPM: 160 pH: 9.86 |

### iv) Addition of Aluminium Hydroxide Solution (iii) into a Solution of (i) and (ii)

Aluminium Hydroxide solution (iii) was added into a solution mixture of (i) and (ii) and additional water (100 g) was added. Resulting mixture was stirred for 1 hour. Turbid solution A was obtained.

TABLE 6

| RPM | Operation | Remarks |
|---|---|---|
| 160 | Addition completed | 100 gm water added |
| 160 | Stirring continue for 1 h | |

Total stirring time after complete addition: 1 h pH: 13.85
Density of mixture: 1.01 weight: 2417.71
Appearance of gel: colloidal solution

### b) Preparation of Aluminosilicate Gel

577 g of fumed silica powder and 5004 g water were slowly added into the solution A under vigorous stirring. Then resultant solution was stirred for 2 hour 5 minutes to obtain a milky white colloidal solution.

The gel so formed (reaction mass) was kept stirred at 30° C. for 3 h.

TABLE 7

| RPM | Operation | Water (Kg) | | pH |
|---|---|---|---|---|
| 160 | Started Addition of fumed silica | — | | 13.85 |
| 200 | 97 gm fumed silica added | 1 | Kg | |
| 200 | 101 gm fumed silica added | 1 | Kg | |
| 280 | 102 gm fumed silica added | 1 | Kg | |
| 390 | 105 gm fumed silica added | 1 | Kg | |
| 450 | 95 gm fumed silica added | 500 | g | |
| 450 | 77 gm fumed silica added | 500 | g | |

| | | | |
|---|---|---|---|
| 460 | Stirring continued | — | |
| 460 | pH of solution checked | — | 13.24 |
| 460 | Stirring stopped | | |
| 460 | Unload the container | | |

Total stirring time after complete addition: 2 h 5 min, pH: 13.24
Density of mixture: 1.05, weight: 7998 g
Appearance of gel: milky white colloidal solution
Total stirring time after complete addition: 2 h 5 min, pH: 13.24

### Example 3: Hydrothermal Crystallization of Aluminosilicate Gel

The reaction mass (hydrous-gel) of aluminosilicate gel was transferred to an autoclave (Make: Flutron, USA, Capacity: 20 L; Type of stirrer: overhead-two stirrer axial stirring Number of Blade: 4).
Weight of Gel added into autoclave=7900 g (7.90 kg)
Final pH of gel 13.24
Close, pack reactor & subject to hydrothermal crystallization 160° C. for 4 days

TABLE 8

| | | Process Temperature | | | |
|---|---|---|---|---|---|
| | | (° C.) | | Pressure | |
| RPM | Operation | SET | | (Kg) | Remark |
| 120 | Start | 160 | 25 | 0 | Control set- |
| | heating up | | | | 170 |
| | to 160° C. | | | | |
| 120 | Temp. | 160 | 147 | 80 psi | |
| | record | | | | |
| 120 | Temp | 160 | 162 | 110 psi | Temp |
| | reached | | | | achieved and |
| | | | | | set |
| 120 | Reading 1 | 160 | 159 | 80 | — |
| 120 | Reading 2 | 160 | 159 | 77 | — |
| 120 | Reading 3 | 160 | 162 | 80 | — |
| 120 | Reading 4 | 160 | 159 | 80 | — |
| 120 | Reading 5 | 160 | 161 | 80 | — |
| 120 | Reading 6 | 160 | 159 | 80 | — |
| 120 | Reading 7 | 160 | 159 | 80 | — |
| 120 | Stopped | 160 | 160 | 80 | — |
| | heating | | | | |
| 120 | Cooling | 24 | 157 | 79 | — |
| | started | | | | |
| 0 | Cooling | | | | Discharged |
| | complete | | | | |

Total stirring time after hydrothermal treatment: 12 hrs, pH = 13.02
Density of slurry: 1.04 weight: 7776 gm
Appearance: white color colloidal solution

### Example 4: Work Up Procedure

a) Filtration: The reaction mixture was filtered and product was washed with De-Mineralized water (5 L+5 L)

TABLE 9

| | Process Temp | |
|---|---|---|
| Operation | (° C.) | Remark |
| Unload the Reaction | RT | Weight = 7776 gm |
| mass | | |
| Filter the Reaction mass | RT | RT |
| | | SS-Nutch filter |
| | | (Width = 24″ × 24″ |
| | | h = 9″, d = 20″) |
| Wash with DM water | | Weight of washing: 6828 gm |
| (5 L + 5 L) | | pH of washed liquid = 12.78 |
| 2^{nd} wash | | Weight of washing = |
| | | pH of washed liquid = 11.46 |
| Wet cake = 810 gm | | |

b) Drying: The product was dried in hot air oven at 120° C. for 5 hours.

TABLE 10

| | SET Temp | Process Temp | |
|---|---|---|---|
| Operation | (° C.) | (° C.) | Remark |
| Dry in Hot Air | 120° C. | 25° C. | (Make: Metalab |
| Oven at 120° C. | | | Capacity: SR no |
| | | | 2269). |
| Maintained for 4- | 120° C. | 120° C. | Temperature |
| 5 hrs | | | achieved |
| Stopped the heating | 120° C. | 20° C. | |
| Weight = 486 gm | | | XRD Pattern |
| | | | SSZ13 |

c) Calcination

The dried product weighing about 486 gm was powdered and then placed (spread) in stainless steel trays. The stainless-steel trays containing product were then placed in a muffle furnace (Make: Energy systems Capacity—200 gm). Temperature of furnace was raised with 1° C./min according to following heating program:

| Temperature | Ramp rate | hold time |
|---|---|---|
| 150° C. | 1º C. | 3 Hr |
| 580° C. | 1º C. | 12 Hr |

TABLE 11

| | SET Temp | Process Temp | |
|---|---|---|---|
| Operation | (° C.) | (° C.) | Remark |
| Calcinations of | — | — | RT to 580° C. as per |
| Weight = 486 | | | above mentioned |
| | | | heating program |
| Completed heating | 580 | 580 | |
| program | | | |
| Weight = 404 gm | | | (XRD Pattern) |

Yield:
1) With respective to total charge=5.03%
2) With respective to silica=70%

### Example 5: Characterization of H-SSZ-13 Catalyst (SiO₂/Al₂O₃-96)

The X-ray diffraction (XRD) patterns of samples were acquired from ‘X’ Pert Pro Phillips diffractometer equipped with Cu, Kα radiation source (operation at 40 kV and 40 mA, λ=A°/nm). The data was recorded in the 2θ range of 5-50°. The morphology and crystal size of samples were obtained using scanning electron microscopy (SEM) on Quant-200 3D instrument operating at 20 kV. The elemental composition of samples analysis was carried out by Energy Dispersive X-ray analysis (EDAX) on Quant-200 3D technique operating at 20 kV. The specific surface area and pore volume analysis were performed on Brunauer-Emmett-Teller (BET) by employing Quantachrome instrument at −196° C. Quantasorb SI automated surface area and pore size analyzer. Prior to analysis, all samples were degassed at 300° C. for 3 h to remove the impure gases adsorbed on catalyst surface.

FIG. 1 describes the XRD pattern of H-SSZ-13 catalyst. In XRD, the first peak (100 plane) is more intense than the normal H-SSZ-13.

FIG. 2 describes FESEM of H-SSZ-13 catalyst. FESEM observed cubical uniform morphology in the range of 2-2.5-micron size.

### Example 6: 2-Methoxyethanol (MME)/Ethylene Glycol (EG) to 1,2-Dimethoxyethane (DME)/Diethoxy ethane (DEE)

### A. Typical Batch Reaction Procedure (Entry 1 of Table 1)

The catalytic conversion of 2-methoxyethanol was performed in a 100 mL stirred SS316 reactor run in a batch mode. The typical catalytic run involves, 18.92 mL of reaction mixture with stoichiometric quantity of 2-Methoxyethanol (7.65 gm) and Methanol (11.27 gm) (1:3.5 of 2-methoxyethanol: Methanol), catalyst (H-SSZ13) loading (0.53 gm) (7% with respect to 2-Methoxyethanol), 210° C., 120 rpm (revolution per minute) for 5 hours. After the completion of reaction, the reactor was cooled down naturally and catalyst was separated by filtration. The reaction products were analyzed by GC-FID with 30 m length HP-5 column. Similar experimental procedures were followed for other experiments in batch mode.

### B. Typical Continuous Reaction Procedure (Entry 23 of Table 1)

The catalytic conversion of 2-methoxyethanol in a continuous mode was performed in 30 cc fixed bed reactor system. HSSZ-13 (SiO₂/Al₂O₃ ratio of 96) was formulated with 20% Alumina binder and converted in to 2 mm×2 mm extrudates. 10 gm of this extrudates HSSZ13 catalyst was loaded at centre of the reactor sandwiched between porcelain beads. The catalyst was activated at 350° C. for 5 h in presence of nitrogen as a carrier gas. After activation, the temperature was reduced to desired temperature (215° C.) in presence of nitrogen. Then nitrogen pressure at 5 bar was generated by continuing nitrogen flow at 50 ml/min. At 215° C., 5 bar nitrogen pressure, the feed mixture of 2-methoxyethanol+Methanol in a molar ratio of 1:3 and WHSV of total mixture to 0.7h-1 was set. After regular time interval of every one hour, the sample was collected and was analyzed by GC as mentioned above. Similar experimental procedure was followed for other continuous experiments.

### Advantages of the Invention

Highest selectivity of 1,2-dimethoxyethane achieved
Catalyst can be used in batch as well as in fixed bed continuous operation.
Catalyst is reusable in batch as well as in fixed bed continuous operation.

1-10. (canceled)

11. A zeolite catalyst H-SSZ-13, characterized by a cubical morphology and having a pore diameter from 0.5 μm to 0.6 μm, a pore volume from 0.2 cc/g to 0.3 cc/g, a surface area from 500 m²/g to 700 m²/g, and a SiO₂/Al₂O₃ ratio from 30 to 200.

12. A process for preparing the zeolite catalyst of claim 11, the process comprising:
(i) hydrothermally crystallizing a gel formed by fumed silica, aluminum hydroxide, sodium hydroxide, N,N,N-trimethyladamantan-1-aminium hydroxide and water by heating at from 100° C. to 200° C. at a pressure from 70 psig to 120 psig for a 4 days to 9 days to obtain a slurry;
(ii) filtering the slurry obtained in (i), followed by drying at from 100° C. to 120° C. for 4 hours to 5 hours to obtain a dried slurry; and
(iii) calcining the dried slurry obtained in (ii) at 500° C. to 600° C. for 10 hours to 14 hours to afford the zeolite catalyst.

13. A one-pot process for synthesizing an ether, the process comprising:
reacting a first substrate with a second substrate in a molar ratio from 1:1 to 1:10 in the presence of the zeolite catalyst according to claim 11, at a temperature from 200° C. to 250° C. for 2 hours to 7 hours to afford the ether;
wherein the process is carried out in a batch or a fixed-bed continuous operation or in a continuous stirred tank reactor.

14. The process of claim 13, wherein:
the first substrate is an alcohol selected from the group consisting of ethylene glycol, propylene glycol, 2-methoxyethanol, and 2-ethoxyethanol; and
the second substrate is an alcohol selected from the group consisting of methanol, ethanol, propanol, and octanol.

15. The process of claim 13, wherein the ether is selected from 1,2-dimethoxyethane or diethoxyethane.

16. The process of claim 13, wherein selectivity of the ether is from 30% to 100% and conversion of the substrate is from 20% to 90%.

17. The process of claim 13, wherein:
the process is carried out in a fixed-bed continuous operation;
a binder is used in the fixed bed continuous operation;
content of the binder with respect to the catalyst from 0.1% to 50%; and
the binder is selected from alumina, silica, or mixture thereof.

18. The process of claim 13, wherein:
the process is carried out in a fixed-bed continuous operation;
the catalyst is shaped as an extrudate, a pellet, or a tablet; and
the catalyst in a continuous operation has a size from 1 mm×1 mm to 5 mm×5 mm; and
the catalyst is recyclable.

19. The process of claim 13, wherein the process is carried out in a fixed-bed continuous operation, in which a weight hourly space velocity with respect to the first substrate is from 0.1 hours⁻¹ to 3 hours⁻¹ and a nitrogen pressure is from 1 bar to 10 bar.

20. The process of claim 13, wherein the process is carried out in a batch operation with a loading of the catalyst from 2% to 10%.
